(19) United States
(12) Patent Application Publication
(10) Pub. No.: US 2018/0270513 A1
(43) Pub. Date: Sep. 20, 2018

(54) SYSTEMS AND METHODS OF MULTI-DATA-COMPONENT VIDEO

(71) Applicant: MNM, LLC, Portland, OR (US)

(72) Inventor: Robert R. Lazzara, Portland, OR (US)

(21) Appl. No.: 15/925,634

(22) Filed: Mar. 19, 2018

Related U.S. Application Data
(60) Provisional application No. 62/472,787, filed on Mar. 17, 2017.

(51) Int. Cl. H04N 21/2365; G06T 19/00; G16H 80/00

(52) U.S. Cl. CPC H04N 21/2365; G16H 80/00; G06T 19/006

(57) ABSTRACT

A method of combining multiple related video data includes acquiring a first video-data stream, acquiring a second video-data stream, combining the first and the second video-data stream; and displaying the first and second video-data stream as a single video-data stream. The method further includes acquiring and combining a third video-data stream to display the single video-data stream including the third video-data stream. The first video-data stream may be a video-data stream of patient interactions by healthcare providers, and the second video-data stream may be a video-data stream of a physician-delivered didactic presentation. The third video-data stream may take the form of a virtual-reality data stream or an augmented-reality data stream, with or without 360-degree immersion.

### CROSS-REFERENCE TO RELATED APPLICATIONS

This application claims the benefit under 35 U.S.C. § 119(e) of U.S. Provisional Patent Application Ser. No. 62/472,787, filed Mar. 17, 2017 and entitled “SYSTEMS AND METHODS OF MULTI-DATA-COMPONENT VIDEO”, the disclosure of which is herein incorporated by reference.

### FIELD OF INVENTION

The present invention relates to systems and methods for combining multiple video streams.

### BACKGROUND OF THE INVENTION

Conventional methods of imparting scientific knowledge, such as medical knowledge, involve giving lectures or demonstrations to students, and publishing textbooks and articles. The demonstrations may include a video presentation in which a speaker discusses a given topic and there are illustrations that support the speaker's discussion.

### SUMMARY OF THE INVENTION

The invention includes a system and a method of combining multiple related video data. The system and method includes suitable structure, such as commercially available video cameras and display equipment, for acquiring a first video-data stream; acquiring a second video-data stream; combining the first and the second video-data stream; and displaying the first and second data stream as a single video-data stream. The system and methods also includes acquiring a third or additional data streams and displaying them with the first and second ones.

### BRIEF DESCRIPTION OF THE DRAWING

The foregoing summary, as well as the following detailed description of various embodiments, is better understood when read in conjunction with the drawing provided herein. For the purpose of illustration, there is shown in the drawing an exemplary embodiments. However, the presently disclosed subject matter is not limited to the specific methods and instrumentalities disclosed.

FIG. 1 is a flow chart illustrating a method for system and a method of combining multiple related video data according to the invention.

### DETAILED DESCRIPTION OF INVENTION

In an embodiment of the invention, multiple related videos of multiple types are combined in a single video format. The first video out of the multiple videos may be a video that is a filmed patient interaction by healthcare providers or lay person. The second video out of the multiple videos may be a video of a physician delivered didactic presentation. The third video out of the multiple videos may be a video of virtual/augmented reality content i.e. 2D/3D content, with or without 360 degrees immersion technology. Additional video outputs could be added.

All three or more videos are then combined together by a suitable processor to generate and display a single video-data stream on a video display unit. In an embodiment of the invention, only first and second videos are combined for generating the single video-data stream. In another embodiment, the first second and third videos are combined for generating the single video-data stream. In another embodiment, more than three videos are combined for generating the single video-data stream.

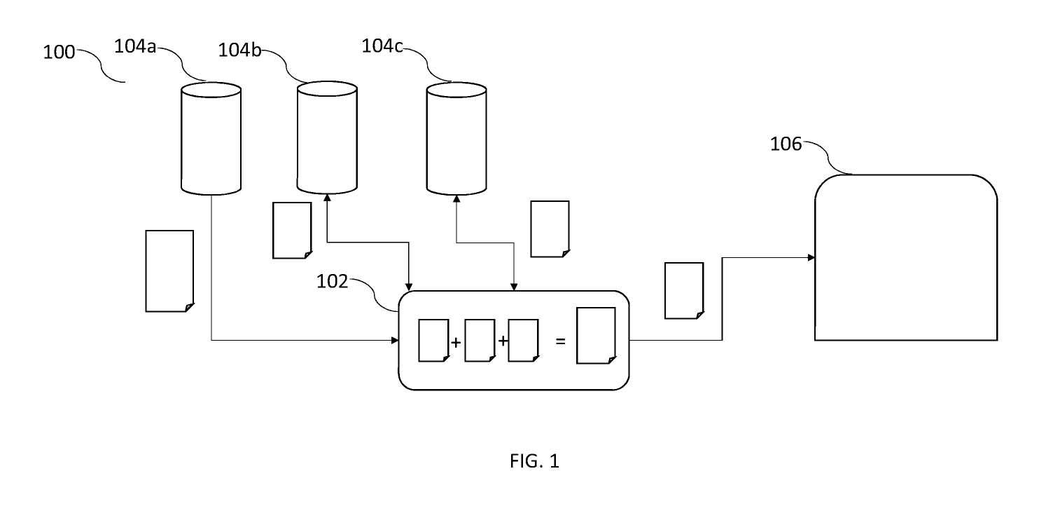

Referring to FIG. 1, illustrating a block diagram of a system 100 that combines multiple related videos to generate a combined video-data stream to display. The system 100 includes a video combiner 102 connected to various video sources 104a, 104b, 104c. The video combiner 102 receives an input of a first type of video from 104a. The video combiner 102 analyzes the video from 104a and fetches a related video from each of the video databases 104b, and 104c.

After, the videos from 104a, 104b, and 104c has been received, the video combiner 102 then combines these three videos and further generates a single combined video-data stream. This single video-data stream can be then displayed on any of a display device 106 like, a television, a monitor, a mobile phone, a laptop, a tablet computer, etc.

In another embodiment of the invention, the video combiner 102 only receives three related videos from each of the video databases 104a, 104b, and 104c and then combines the three video-data streams to generate a single video-data stream to be displayed on a display device 106.

The invention includes displaying a combination of all three video streams in a rich media format that also includes standard computer graphics and video production format. This production format surpasses standard methods of knowledge retention and knowledge transfer for education.

The flowchart in the FIGURE illustrates the functionality, and operation of possible implementations of systems and methods according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While certain embodiments have been described, these embodiments have been presented by way of example only, and are not intended to limit the scope of the present disclosure. Indeed, the novel methods, devices, and systems described herein may be embodied in a variety of other forms. Furthermore, various omissions, substitutions, and changes in the form of the methods, devices, and systems described herein may be made without departing from the spirit of the present disclosure. The accompanying claims and their equivalents are intended to cover such forms or modifications as would fall within the scope and spirit of the present disclosure.

What is claimed:

1. A method of combining multiple related video data comprising;
acquiring a first video-data stream;
acquiring a second video-data stream;
combining the first and the second video-data stream; and
displaying the first and second data stream as a single video-data stream.

2. The method of claim 1, wherein the method further includes acquiring and combining a third video-data stream to display the single video stream including the third video-data stream.

3. The method of claim 1, wherein the first video-data stream is a video-data stream of patient interactions by healthcare providers.

4. The method of claim 1, wherein the second video-data stream is a video-data stream of a physician-delivered didactic presentation.

5. The method of claim 1, wherein the third video-data stream is a virtual-reality data stream with 360-degree immersion.

6. The method of claim 1, wherein the third video-data stream is an augmented-reality data stream with 360-degree immersion.

7. The method of claim 1, wherein the third video-data stream is a virtual-reality data stream without 360-degree immersion.

8. The method of claim 1, wherein the third video-data stream is an augmented-reality data stream without 360-degree immersion.
